(19) United States
(12) Patent Application Publication
(10) Pub. No.: US 2014/0045741 A1
(43) Pub. Date: Feb. 13, 2014

(54) Method Of Treating An Ocular Disease And Compositions Effective For Treating An Ocular Disease

(71) Applicant: Howard University, Washington, DC (US)

(72) Inventors: Pradeep K. Karla, Washington, DC (US); Harpal S. Mangat, Washington, DC (US)

(73) Assignee: HOWARD UNIVERSITY, Washington, DC (US)

(21) Appl. No.: 14/059,035

(22) Filed: Oct. 21, 2013

Related U.S. Application Data
(63) Continuation-in-part of application No. PCT/US2013/032187, filed on Mar. 15, 2013.
(60) Provisional application No. 61/636,143, filed on Apr. 20, 2012.

(51) Int. Cl. A61K 38/13

(52) U.S. Cl. CPC A61K 38/13
USPC 514/1.2

(57) ABSTRACT

Methods and compositions are provided for treating an ocular disease in a subject in need thereof by increasing the bioavailability of a drug in the subject's eye. In one aspect, the ocular disease is at least one of diabetic retinopathy, macular degeneration, endopthalmitis, glaucoma, cataracts, ocular herpes. The methods and compositions provided herein include an efflux transporter inhibitor and a drug effective for treating the ocular disease. The efflux transporter inhibitor is effective to reduce the efflux of the drug through at least one of P-glycoprotein (Pgp), breast cancer resistant protein (BCRP), and multidrug resistant associated protein 1-9 (MRP1-9).

### CROSS REFERENCE TO RELATED APPLICATION

This application is a continuation-in-part of PCT/US2013/032187 filed Mar. 15, 2013 which claims the benefit of U.S. Provisional Application No. 61/636,143, filed Apr. 20, 2012, both of which are incorporated herein by reference in their entirety.

### FIELD

Methods and compositions are described for the treatment of ocular diseases in humans. More particularly, the methods and compositions include efflux transporter inhibitors and ocular drugs where the drugs are effective for the treatment of ocular diseases which include but are not limited to diabetic retinopathy, macular degeneration, endopthalmitis, glaucoma, cataracts, ocular herpes and other ocular diseases which can be treated to effect new blood vessels in the eye through angiogenesis of new blood vessels via medication.

### BACKGROUND

Many pharmacologic compositions for the treatment of ocular diseases are topically applied to the surface of the eye in the form of eye drops, gels, ointments or suspensions. The effectiveness of these pharmacologic compositions is limited, at least in part, by natural barriers present in the eye. It has been reported that bioavailability of ocular drugs within the eye is generally about 1 to 10 percent for topically administered drugs. For instance, the cornea is a known primary barrier for topical ocular drug delivery to the anterior segment of the eye. The primate cornea has five layers: epithelium, bowman's layer, stroma, descement's membrane, and endothelium. The epithelium includes tightly packed, stratified cells which form a major barrier for drug permeation. It has been found that tight junctions and an array of drug efflux transporters in the epithelium are important factors for the poor delivery of ocular drugs. Efflux transporters play an important role in conferring drug resistance by pumping out the drug compounds, such as antimicrobials, antibiotics, beta blockers, carbonic anhydrase inhibitors, prostaglandin analogues, miotics, and sympthaomimetics by an energy-dependent mechanism away from the target site.

Efflux transporters identified in the eye or cornea include P-glycoprotein 1 (Pgp), breast cancer resistant protein (BCRP), and multidrug resistant associated proteins 1-9 (MRPs 1-9). While not wishing to be bound by theory, it is presently believed that MRPs are considered to play a significant role in drug efflux compared to Pgp and BCRP in the eye, and often, more specifically the cornea. The presence of these efflux transporters in ocular tissue and/or in or on the cornea have been found to confer drug resistance to a variety of topically applied drugs. The presence of MRPs, Pgp and BCRP efflux pumps in or the eye or cornea imply the likelihood of drug resistance to a wide range of ocular drugs. Ocular and/or corneal efflux pumps can also act in an additive manner to efflux a wider range of drug molecules, forming a very strong physical barrier to ocular drug delivery.

MRPs are proteins which effect drug resistance in mammalian cells. By way of example, they include multidrug resistant protein 2, also known as multispecific organic transporter 1 (CMOAT), which is encoded by the human ABCC2 gene; and multidrug resistant protein 4 which is encoded by the human ABCC4 gene.

Pgp belongs to the ATP-binding cassette (ABC) family of transporters which use adenosine triphosphate (ATP) as an energy source. Pgp is a glycoprotein that is encoded in humans by the ABCB 1 gene and also is known as ATP-binding cassett Subfamily B member 1. This efflux transporter has two transmembrane sites embedded in a lipid bilayer of a cell membrane. Each transmembrane site includes six transmembrane domains. Pgp has two nucleotide binding domains which are also known as ATP binding domains.

MRPs appear to play a major role in drug efflux and the resulting decrease in drug efficacy. MRPs are also classified in the ABC family of transporters and require adenosine triphosphate (ATP) for efflux function. The major structural difference between MRPs and Pgp is the presence in MRPs of an additional transmembrane site in a lipid bilayer, which includes five transmembrane domains. Additionally, another significant difference between the two transporters is the presence in MRP of an amino terminal on the external side of cell membrane.

BCRP was recently identified in human corneal epithelial cells. BCRP also primarily employs ATP for its efflux function. BCRP is referred to as a “half transporter” and has one transmembrane site and one ATP binding site. The transmembrane site has six transmembrane domains embedded in the lipid bilayer.

The relative rate of efflux through the eye or corneal epithelium is governed by multiple factors, such as the drug substrate specificity with the efflux transporter and the extent of expression of a particular efflux transporter compared to other efflux transporters.

The treatment of ocular diseases is significantly limited by the difficulty in delivering effective doses of drugs to the target areas of the eye due to the presence of the efflux transporters. Further, the relatively small proportion of a topically applied pharmacologic composition that reaches the necessary site in the eye for activity often requires administration of high concentrations of the pharmacologic composition, which can lead to a variety of undesirable side effects.

### SUMMARY

Described herein are methods and compositions for treating ocular diseases including diabetic retinopathy, macular degeneration, endopthalmitis, glaucoma, cataracts, ocular herpes and other ocular diseases which can be treated through the creation of new blood vessels in the eye through angiogenesis of new blood vessels in a subject afflicted which such diseases. It has been found that inhibition of the drug efflux transporters in ocular tissue including the corneal epithelium significantly improves bioavailability of many ocular drugs. The methods and compositions provided herein include one or more efflux transporter inhibitors in combination with at least one ocular drug, where the combination of the efflux transporter inhibitor and one at least one ocular drug is not only effective for the treatment of an ocular disease, it increases the effectiveness of the drug by at least 100% when the drug is applied at the same dosage levels at the same rate to treat the same disease at the same disease stage. In other forms, the combination increases the effectiveness of the drug by at least 200%. In another aspect, a lower dosage amount of the drug may be used to achieve the same effectiveness. In this regard, 50% of a standard dose could be used to achieve the same efficacy as a regular full dose. In this aspect, an efflux transporter inhibitor can be incorporated into a treatment regimen to be used in conjunction with an ocular drug. It has not been previously demonstrated that employing drug efflux inhibition is effective to treat ophthalmic disease in humans.

In one aspect, the efflux transporter inhibitor used in the methods and compositions described herein is effective to reduce the efflux of the drug through at least one efflux transporter in ocular tissue including the cornea. In this aspect, the efflux transporter may be selected from the group consisting of P-glycoprotein (Pgp), breast cancer resistant protein (BCRP), and multidrug resistant associated proteins 1-9 (MRP1-9).

In another aspect, a method is provided for treating an ocular disease in a subject in need thereof by increasing the bioavailability of a drug in the subject's eye by mitigating the effects of the efflux transporters described above. The method comprising applying the efflux transporter inhibitor and a therapeutically effective amount of a drug, such as antimicrobials, antibiotics, beta blockers, carbonic anhydrase inhibitors, prostaglandin analogues, miotics, and sympthaomimetics and an efflux transporter inhibitor, such as bevacizumab, itraconazole, caroxyamidotriazole, TNP-470, CM101, IFN-α, IL-12, platelet factor-4, suramin, SU5416, thrombospondin, VEGFR antagonists, angiostatic, steroids+heparin, cartilage-derived Angiogenesis Inhibitory Factor, matrix metalloproteinase inhibitors, angiostatin, endostatin, 2-methoxyestradiol, tecogalan, tetrathiomolybdate, thalidomide, thrombospondin, prolactin, αᵥβ₃ inhibitors, linomide, tasquinimod, and cyclosporine A. The amount of efflux transporter inhibitor applied is in an amount effective to reduce the efflux of the drug through the subject's ocular tissue. Generally, the ratio of the drug to efflux transporter inhibitor should be in the range of from about 5:1 to about 1:5, preferably from about 2:1 to about 1:2. In one approach, the ratio of the drug to efflux transporter inhibitor is about 1:1. By one approach, the drug and efflux transporter inhibitor are applied topically to the subject's eye with a pharmaceutically acceptable carrier such as an ointment or eye drops. In one aspect, the subject is a human.

In a particularly important aspect, a method is provided for the treatment of diabetic retinopathy, macular degeneration, and glaucoma. In this aspect the drug, which can be used in combination with efflux pump inhibitors described above, include beta blockers such as timolol, levobunolol, betaxolol and carteolol; carbonic anhydrase inhibitors such as dorzolamide, timolol and brinzolamide; prostaglandin analogues such as latanoprost, latanoprost plus, timolol, travoprost, bimatoprost and unoprostone; and miotics and sympthaomimetics such as pilocarpine, dipivefrine and brimonidine. Accordingly, a method is provided for treating diabetic retinopathy, macular degeneration, and glaucoma in a subject in need thereof by increasing the bioavailability of a drug in the subject's eye, the method comprising applying a therapeutically effective amount of the drug and an efflux transporter inhibitor, the amount of efflux transporter inhibitor applied in an amount effective to reduce the efflux of the drug from the subject's ocular tissue and cells. By one approach, the drug and efflux transporter inhibitor are applied topically to the subject's eye with a pharmaceutically acceptable carrier such as an ointment or eye drops. In one particular aspect, the efflux transporter inhibitor is Cyclosporine A (Restasis) to treat Endophthalmitis. The amount of efflux transporter inhibitor included in the methods and compositions described herein is effective to reduce the efflux of the drug through the subject's ocular tissue and/or cornea to improve the therapeutic efficacy of the drug when administered in a given quantity by at least 100%, and in some forms, at least 200%, when the drug is applied at the same dosage levels at the same rate to treat the same disease at the same disease stage.

Generally, the time between application of the efflux transporter inhibitor and the drug, the relative amounts of the efflux transporter inhibitor and the drug, and the ratio of the efflux transporter inhibitor to the drug are effective to increase the therapeutic efficacy of the drug for treating the ocular disease as compared to administering the same amount of the drug without the efflux transporter inhibitor. In one aspect, the efflux transporter inhibitor is administered before administration of the drug, such as within minutes of administration of the efflux transporter inhibitor.

A composition for the treatment of an ocular disease in a subject is also described herein. The composition comprises an efflux transporter inhibitor and at least one ocular drug effective for the treatment of, such as antimicrobials, antibiotics, beta blockers, carbonic anhydrase inhibitors, prostaglandin analogues, miotics, and sympthaomimetics; efflux pump inhibitors such as bevacizumab, itraconazole, caroxyamidotriazole, TNP-470 (a synthetic analog of fumagillin, an antibiotic isolated from the fungus Aspergillus fumigatus fresenius with antineoplastic activity, (3R,4S,5S,6R)-5-methoxy-4-[(2R,3R)-2-methyl-3-(3-methyl-2-butenyl)-oxiranyl]-1-oxaspiro [2,5] oct-6-yl(chloroacetyl) carbamate 5-methoxy-4-(2-methyl-3-(3-methyl-2-butenyl)-1-oxaspiro(2,5)oct-6-yl(chloroacetyl) carbamate O-(chloroacetylcarbamoyl)fumagillol), CM101 (a polysaccharide tumor angiogenesis-targeting, complement activating, immune stimulating biological produced fermentatively from Group B Streptococcus), IFN-α (interferon), IL-12 (interleukin), platelet factor-4, suramin (C₅₁H₄₀N₆O₂₃S₆, 8,8′-{Carbonylbis[imino-3,1-phenylenecarbonylimino(4-methyl-3,1-phenylene)carbonylimino]}di(1,3,5-naphthalenetrisulfonic acid)), SU5416 (C₁₅H₁₄N₂O, 1,3-Dihydro-3-[(3,5-dimethyl-1H-pyrrol-2-yl)methylene]-2H-indol-2-one), thrombospondin, VEGFR (vascular endothelial growth factor receptor) antagonists, angiostatic, steroids+heparin, cartilage-derived Angiogenesis Inhibitory Factor, matrix metalloproteinase inhibitors, angiostatin, endostatin, 2-methoxyestradiol, tecogalan, tetrathiomolybdate, thalidomide, thrombospondin, prolactin, αᵥβ₃ inhibitors, linomide, tasquinimod, and cyclosporine A and a pharmaceutically suitable carrier such as an ointment or eye drops suitable for application to ocular tissue. The drug is in a therapeutic amount for the treatment of the ocular disease. The relative amounts of the efflux transporter inhibitor and the drug, as well as the ratio of the efflux transporter inhibitor to the drug, in the composition are effective for increasing a therapeutic efficacy of the drug for treating the ocular disease as compared to administering the same amount of the drug without the efflux transporter inhibitor. In one aspect, the ocular disease is diabetic retinopathy, macular degeneration, endopthalmitis, glaucoma, cataracts, keratoconus, ocular herpes and other ocular diseases which can be treated through the creation of new antimicrobials. In another aspect, the drug may be one which is effective for the treatment of the latter diseases. In another aspect, the drugs include beta blockers, carbonic anhydrase inhibitors, prostaglandin analogues, miotics, sympthaomimetics ceftazidine, ciprofloxacin, vancomycin, or moxifloxacin. In yet another aspect, the efflux transporter inhibitor is cyclosporine A. In another aspect, the composition is effective for the treatment of endophthalmitis.

### DETAILED DESCRIPTION

Described herein are methods and compositions for treating an ocular disease in a subject by increasing the ocular bioavailability of a drug by modulating the efflux of the drug through the subject's eye and/or related tissue and/or fluid. It has been discovered that there are significant benefits in drug efficacy, and also treatment of certain diseases, by incorporating drug efflux transporter modulators in ophthalmic compositions and/or treatment regimens. In some approaches, modulation of the efflux transporters is by inhibition. It has been found that inhibition of the drug efflux transporters in the corneal epithelium can significantly improve bioavailability of many ocular drugs, thereby significantly increasing the efficacy of the ocular drug. Conventional ocular therapies do not involve use of efflux transporter inhibitors in conjunction with other medicaments.

Endophthalmitis is an inflammation and infection of tissues in the eye, often as a result of microbial infection after surgery or eye trauma. The inflammation generally affects the vitreous fluid in the center of the eye but can also affect neighboring areas of the eye responsible for vision. Symptoms of endopthalmitis include, for example, blurred vision, eye pain, and redness. In many instances, the loss of vision is irreversible. Endophthalmitis is typically treated with intraocular antibiotics and anti-inflammatory agents. In severe endophthalmitis, blindness can occur despite treatment.

By one approach, a method is provided for treating an ocular disease in a subject in need thereof by increasing the bioavailability of a drug in the subject's eye, the method comprising applying a therapeutically effective amount of a drug and an efflux transporter inhibitor to the subject's eye, the efflux transporter inhibitor applied in an amount effective to reduce the efflux of the drug through the portions of a subject's eye. By one approach, the drug and efflux transporter inhibitor are applied topically to the subject's eye. In one aspect, the subject is a human. In one aspect, the ocular disease is endophthalmitis. In one aspect the ocular disease is diabetic retinopathy, macular degeneration, endopthalmitis, glaucoma, cataracts, ocular herpes and other ocular diseases which can be treated to effect new blood vessels in the eye through angiogenesis of new blood vessels via medication

By another approach, a method is provided for treating endopthalmitis in a subject in need thereof by increasing the bioavailability of a drug in the subject's eye, the method comprising applying a therapeutically effective amount of a drug and an efflux transporter inhibitor to a subject's eye, the amount of efflux transporter inhibitor applied in an amount effective to reduce the efflux of the drug through the subject's eye. By one approach, the drug and efflux transporter inhibitor are applied topically to the subject's eye. In one particular aspect, the efflux transporter inhibitor is cyclosporine A and the drug comprises an antimicrobial. In one aspect, the subject is a human.

By one approach, the time between application of the efflux transporter inhibitor and the drug, the relative amounts of the efflux transporter inhibitor and the drug, and the ratio of the efflux transporter inhibitor and the drug are effective to increase the therapeutic efficacy of the drug for treating the ocular disease as compared to administering the same amount of the drug without the efflux transporter inhibitor.

At least in some approaches, the drug and efflux transporter inhibitor are applied to the subject's eye at substantially the same time. By “substantially the same time” is meant within about 10 minutes, in another aspect within about 5 minutes, in another aspect within about 1 minute, and in another aspect within about 0.5 minutes.

As used herein, the term “treating” refers to an intervention performed to alter the pathology of, and thereby substantially alleviate or reduce in severity, an ocular disease or condition, including one or more symptoms of such disease or condition in a subject. As used herein, the term “subject” includes mammals and specifically includes humans. Veterinary applications are also contemplated. Accordingly, “treating” refers to both therapeutic treatment and prophylactic measures. The related term “treatment,” as used herein, refers to the act of treating a symptom, disease or condition. Those in need of treatment include subjects already having an ocular disorder or ocular disease. By some approaches, the subject is in recognized need of treatment. For example, the subject may exhibit symptoms of ocular disease. In one aspect, the subject has been diagnosed by a medical professional as having an ocular disease or displaying symptoms of an ocular disease. In one aspect, the ocular disease is endopthalmitis. In another aspect, the ocular disease or condition is glaucoma, cataracts, macular degeneration, ocular herpes, diabetic retinopathy, macular degeneration, and other ocular diseases which can be treated to effect new blood vessels in the eye through angiogenesis of new blood vessels via medication.

As used herein, the terms “therapeutically effective amount” or “effective amount” refer to the amount of drug and/or efflux transporter modulator required to confer a biological or meaningful patient benefit, such as the biological or medical response or improvement sought by a medical doctor or other medical professional. In one aspect, the terms “therapeutically effective amount” or “effective amount” are intended to mean the amount of drug and/or efflux transporter modulator that will bring about a biologically meaningful improvement in the subject's ocular disorder, symptom, or disease. Doses that exhibit large therapeutic indices are preferred. Effective amounts may vary, as recognized by those skilled in the art, depending, for example, on route of administration, dosage form, inclusion of additional active agents, as well as age, weight, sensitivity, and health of the subject.

As used herein, the term “efflux transporter inhibitor” means a chemical compound, protein, peptide, or other molecule that is effective to stop or reduce extrusion of a drug outside the cell via at least one efflux transporter in the subject's cornea. In some approaches, the efflux transporter inhibitor is effective to stop or reduce extrusion of a drug via at least one efflux transporter selected from the group consisting MRPs, BCRP, and Pgp of the corneal epithelium. Examples of efflux transporter inhibitors include, for example, MK-571 (C₂₆H₂₆ClN₂O₃S₂. Na; a specific MRP inhibitor sold by Biomol International L.P. (PA, USA)), ketoconazole (a specific Pgp inhibitor), GF120918 (a specific Pgp inhibitor marketed as Elacridar by Santa Cruz Biotechnology), indomethacin, PGP-4008, bimatoprost (marketed as LUMIGAN® by Allergan), latanoprost (marketed as XALATAN® by Pfizer), sulfinpyrazone (a MRP5 modulator), and cylosporin-A (a Pgp inhibitor marketed as RESTASIS® by Allergan). Advantageously, cyclosporin-A may be used in the methods and compositions described herein as it is FDA approved for ocular use. Very strong substrates for the efflux transporters can act as inhibitors. Therefore, additional efflux transporter inhibitors can be designed and identified by one of ordinary skill in the art.

It has been found that efflux transporters (such as MPR and Pgp) sometimes act in conjunction to efflux certain drugs, and the combined activity of the efflux transporters forms a strong physical barrier against ocular drug delivery. In some approaches, use of a combination of efflux transporter inhibitors can result in an at least additive increase in uptake of drugs. While not wishing to be limited by theory, it is presently believed that MRP plays a more significant role compared to Pgp in ocular drug efflux of macrolides. Also not wishing to be limited by theory, it is presently believed that MRP4/MRP5 transporters play a significant role in the efflux of nucleoside and nucleotide analogues. It has also been demonstrated that efflux inhibitor GF120918 interacts with both BCRP and Pgp.

The efflux transporter inhibitor should be selected in conjunction with the drug selected for the particular treatment regimen. For example, it is known that erythromycin is a good substrate for MRP efflux but not a good substrate for Pgp efflux. Therefore, an efflux transporter inhibitor would be selected that inhibits MRP efflux. Selection of an efflux transporter inhibitor that inhibits Pgp efflux but not MRP efflux would not be expected to provide the desired clinical benefit. Also, for example, MK-571 is non-specific inhibitor for MRP1-9 but not for Pgp and BCRP. Therefore, MK-571 would be used in conjunction with a drug that is a substrate for MRP1-9 but not for Pgp and BCRP. As another example, MRP2 and MRP5 are involved in acyclovir efflux so an MRP inhibitor, such as MK-571, would be appropriate for treatment with acyclovir. Two widely used glaucoma drugs, bimatoprost and latanoprost, are both substrates for MRP5 and can suppress efflux of drugs administered therewith that are also substrates of MRP5.

In one aspect, the efflux transporter inhibitor is applied in an amount effective to reduce the efflux of the drug through the subject's eye for a period of time sufficient for the administered drug to have clinical benefit to the patient.

As used herein, “drug” comprises at least one active ingredient, including, for example, compound, protein, peptide, or prodrug compound, that is effective to ameliorate or reduce one or more symptoms of an ocular disease. In one aspect, the efficacy of the drug is substantially increased when used in conjunction with an efflux transporter inhibitor. In one aspect, the drug is a substrate of at least one efflux transporter selected from the group consisting of MRPs, BCRP, and Pgp. In some approaches, the active ingredient includes at least one of an antimicrobial (including, for example, antibiotic, antifungal, and antiviral compounds). Exemplary antimicrobials include, for example, amoxicillin, ciprofloxacin, moxifloxacin, cephalexin, vancomycin, ceftazidime, amphotericin, doxycycline, tobramycin, amikacin, gentamicin, clindamycin, cefazolin, ceftazimide, ceftriaxone, cefotaxime, cloramphenicol, erythromycin, oflaxacin, gatifloxacin, acyclovir (also called acycloguanosine and marketed as ZOVIRAX® by GlaxoSmithKline LLC), valacyclovir (VALTREX) and combinations thereof.

When the ocular disorder being treated is endophthalmitis, ameliorating or reducing one or more symptoms of endophthalmitis includes, for example, reducing eye redness, eye pain, and improving blurred or lost vision.

In some approaches, the methods and/or compositions described herein may further include a second active ingredient in addition to the antimicrobial active agent and efflux transporter inhibitor. In one aspect, the second active ingredient may include, for example, a steroid or anti-inflammatory agent. Steroids useful in the methods and compositions described herein include, for example, hydrocortisone, fluromethalone (FML), fluromethalone acetate (FLAREX®), prednisolone sodium phosphate (marketed as Predsol), prednisolone actetate (PRED FORTE®), and dexamethasone (MAXIDEX™). The second active ingredient can be provided in the same or different drug as the antimicrobial active agent.

By some approaches, the drug can include both an antimicrobial and a second active ingredient. By other approaches, it is contemplated that the antimicrobial and second active agent are provided in separate compositions and are separately applied to the eye. In some approaches, at least two of the group consisting of antimicrobial, steroid, and anti-inflammatory are used in combination with an efflux transporter inhibitor.

In one aspect, the drug may be applied directly to a target tissue, in one aspect the subject's eye, cornea, or to a surrounding fluid or tissue. By some approaches, administration to the desired location may be by topical application.

The drug can be prepared in a variety of forms. For example, a liquid formulation can be prepared, such as, for example, in the form of a solution, emulsion, or suspension in a non-toxic, pharmaceutically-acceptable carrier. In another aspect, the drug may be a powder or lyophilisate that is reconstituted with a solvent prior to use. In yet another aspect, the formulation may be in the form of an emulsion or liquid concentrate that is suitable for dilution prior to administration. Exemplary pharmaceutically-acceptable carriers include saline, buffered saline, isotonic saline, Ringer's solution, dextrose, sterile water, deionized water, glycerol, ethanol, 5% dextrose in water, and combinations thereof.

The drug may comprise a variety of optional ingredients. For example, the topical formulation may include ingredients such as but not limited to preservatives, lubricant, stabilizer, colorant, diluent, isotonic agent, pH modifier, buffer, excipient, and the like and additional active ingredients, if desired. In one aspect, any additional ingredients included in the composition should not negatively impact the stability of the active ingredient(s) in the drug.

The treatment regimen for the ocular disease can vary depending on the particular needs of the subject. For example, the dose and frequency of administration of the drug and efflux transporter inhibitor may depend in part on the age of the subject and severity of ocular disease. By way of non-limiting illustration, the combination of drug and efflux transporter inhibitor may be applied at least once daily. By another approach, the combination of drug and efflux transporter inhibitor may be applied at least twice a day. Some subjects may benefit from regular application of the formulation, such as for at least about 3 days, in another aspect at least about 10 days. A shorter or longer treatment regimen may be used, if desired.

The combination therapy of drug and efflux transporter inhibitor can be administered to a subject for improved ocular drug bioavailability and therapeutic efficacy for the treatment of other ocular diseases, including, for example, diabetic retinopathy, macular degeneration, glaucoma, cataracts, ocular herpes and other ocular diseases which can be treated to effect new blood vessels in the eye through angiogenesis of new blood vessels via medication.

Advantages and embodiments of the method and compositions described herein are further illustrated by the following example; however, the particular conditions, processing schemes, compositions, and amounts thereof recited in these examples, as well as other conditions and details, should not be construed to unduly limit this method. All percentages are by weight unless otherwise indicated. All references listed herein are incorporated herein by reference in their entireties.

### EXAMPLE

### Combination Therapy Treatment Method

A uniocular 87 year old female subject was diagnosed with severe endophthalmitis in her only functioning eye. The subject presented with opacity behind the lens. The subject was non-responsive to the initial treatment regimen with no light perception. The subject was treated with an aggressive treatment regimen of topical antibiotics Ceftazidime eye drops, Vancomycin eye drops and steroid Prednisolone eye drops every two hours. The subject showed no improvement over the next two days (initial treatment regimen that did not prevent a drop in vision from reading newspapers to no light perception in two days). The subject was at risk of total vision loss in the diseased visually functioning eye.

Cyclosporin A (sold as RESTASIS® from Allergan, Inc.) was added to the treatment regimen and was administered prior to administering the antibiotic and steroid eye drops. A 16 mg dose of dexamethasone I.M. injection (8 mg in each arm) was given to the subject to decrease inflammation and the subject was asked to administer Gatifloxacin (500 mg table/day for seven days), which has been shown to cross the blood-retina barrier. The treatment regimen was administered with the hypothesis that it would result in inhibition of drug efflux barrier on the blood ocular barrier resulting in elevated posterior segment drug concentrations.

The subject's visual acuity improved dramatically by the next day. The next day the subject was given antibiotic Ceftriaxone (500 mg I.M.) injection and told to continue to use the cyclosporine A antibiotic/steroid eye drops. The Ceftriaxone was added to the regimen to increase the aggressiveness of the therapy with the goal of preventing or eliminating any traces of infection in the retina, which can result in irreversible blindness. Further, it was hypothesized that inhibition of drug efflux barrier on the blood ocular barrier will result in elevated vitreous concentration of Ceftriaxone antibiotic.

Within three to four days after addition of cyclosporine A of the regimen, the subject showed significant signs of improving vision with no visible signs of ocular inflammation and a week later showed a marked improvement in vision. Within the next week, the patient regained 20/50 vision with no signs of endophthalmitis or regression of visual acuity. The infection cleared and the subject's visual acuity was good upon reexamination nearly a year after the treatment. Now she can read fine print and is reading the newspaper again. The restoration of the subject's vision is of high clinical significance because it is believed that less than 5 percent of endopthalmitis patients report vision restoration to this extent.

While this disclosure has been particularly described with specific reference to particular processes and embodiments, it will be appreciated that various alterations, modifications, and adaptations may be based on the present disclosure, and are intended to be within the spirit and scope of the disclosure as defined by the following claims.

What is claimed is:

1. A method for treating an ocular disease in a human subject in need thereof by increasing the bioavailability of a drug in the subject's eye, the method comprising applying a therapeutically effective amount of a drug and an efflux transporter inhibitor, the amount of efflux transporter inhibitor applied in an amount effective to reduce the efflux of the drug through the subject's cornea.

2. The method according to claim 1, wherein the drug and efflux transporter inhibitor are topically applied to the subject's eye.

3. The method according to claim 1, wherein the efflux transporter inhibitor is effective to reduce the efflux of the drug through at least one efflux transporter selected from the group consisting of P-glycoprotein (Pgp), breast cancer resistant protein (BCRP), and multidrug resistant associated protein 1-9 (MRP1-9).

4. The method according to claim 1, wherein the drug is effective to treat at least one of diabetic retinopathy, macular degeneration, endopthalmitis, glaucoma, cataracts, ocular herpes and ocular diseases which can be treated to effect new blood vessels in the eye through angiogenesis of new blood vessels.

5. The method according to claim 1, wherein the drug is at least one of antimicrobial, antibiotic, beta blocker, carbonic anhydrase inhibitor, prostaglandin analogue, miotic, and sympthaomimetic.

6. The method according to claim 1, wherein the ocular disease is endophthalmitis.

7. The method according to claim 1, wherein the efflux transporter inhibitor is at least one of bevacizumab, itraconazole, caroxyamidotriazole, TNP-470, CM101, IFN-α, IL-12, platelet factor-4, suramin, SU5416, thrombospondin, VEGFR antagonists, angiostatic, steroids+heparin, cartilage-derived Angiogenesis Inhibitory Factor, matrix metalloproteinase inhibitors, angiostatin, endostatin, 2-methoxyestradiol, tecogalan, tetrathiomolybdate, thalidomide, thrombospondin, prolactin, αᵥβ₃ inhibitors, linomide, tasquinimod, and cyclosporine A.

8. The method according to claim 1, wherein the drug is at least one of timolol, levobunolol, betaxolol and carteolol, dorzolamide, brinzolamide, latanoprost, latanoprost plus, travoprost, bimatoprost, unoprostone, pilocarpine, dipivefrine and brimonidine.

9. The method according to claim 1, wherein the efflux transporter inhibitor and drug are administered to ocular tissue at substantially the same time.

10. A method for treating at least one of diabetic retinopathy, macular degeneration, endopthalmitis, glaucoma, cataracts, ocular herpes in a subject in need thereof by increasing the bioavailability of a drug in the subject's eye, the method comprising administering to the subject's eye an efflux transporter inhibitor and a therapeutically effective amount of a drug which is effective for the treatment of at least one of diabetic retinopathy, macular degeneration, endopthalmitis, glaucoma, cataracts, ocular herpes, the amount of efflux transporter inhibitor applied in an amount effective to reduce the efflux of the drug through the subject's eye tissue and increase the effectiveness of the drug by at least 100% when the drug is applied at the same dosage levels at the same rate to treat the same disease at the same disease stage.

11. The method according to claim 10, wherein the efflux transporter inhibitor is at least one of bevacizumab, itraconazole, caroxyamidotriazole, TNP-470, CM101, IFN-α, IL-12, platelet factor-4, suramin, SU5416, thrombospondin, VEGFR antagonists, angiostatic, steroids+heparin, cartilage-derived Angiogenesis Inhibitory Factor, matrix metalloproteinase inhibitors, angiostatin, endostatin, 2-methoxyestradiol, tecogalan, tetrathiomolybdate, thalidomide, thrombospondin, prolactin, αᵥβ₃ inhibitors, linomide, tasquinimod, and cyclosporine A.

12. The method according to claim 10, wherein the efflux transporter inhibitor and drug are administered at substantially the same time.

13. The method according to claim 10, wherein the drug and efflux transporter inhibitor are topically applied to the subject's eye.

14. The method according to claim 10, wherein the efflux transporter inhibitor is effective to reduce the efflux of the drug through at least one efflux transporter selected from the group consisting of P-glycoprotein (Pgp), breast cancer resistant protein (BCRP), and multidrug resistant associated protein 1-9 (MRP1-9).

15. The method according to claim 10, wherein the drug is at least one of antimicrobial, antibiotic, beta blocker, carbonic anhydrase inhibitor, prostaglandin analogue, miotic, and sympthaomimetic.

16. The method according to claim 10, wherein the drug is at least one of timolol, levobunolol, betaxolol and carteolol; dorzolamide, brinzolamide, latanoprost, latanoprost plus, travoprost, bimatoprost, unoprostone, pilocarpine, dipivefrine and brimonidine.

17. A composition for the treatment of an ocular disease in a subject, the composition comprising:
an ocular drug selected from the group consisting of antimicrobial, antibiotic, beta blocker, carbonic anhydrase inhibitor, prostaglandin analogue, miotic, and sympthaomimetic; and
an efflux transporter inhibitor is at least one bevacizumab, itraconazole, caroxyamidotriazole, TNP-470, CM101, IFN-α, IL-12, platelet factor-4, suramin, SU5416, thrombospondin, VEGFR antagonists, angiostatic, steroids+heparin, cartilage-derived Angiogenesis Inhibitory Factor, matrix metalloproteinase inhibitors, angiostatin, endostatin, 2-methoxyestradiol, tecogalan, tetrathiomolybdate, thalidomide, thrombospondin, prolactin, αᵥβ₃ inhibitors, linomide, tasquinimod, and cyclosporine A,
the amount of efflux transporter inhibitor in an amount effective to reduce the efflux of the drug through the subject's eye tissue and increase the effectiveness of the drug by at least 100% when the drug is applied at the same dosage levels at the same rate to treat the same disease at the same disease stage, and the amount of drug effective to reduce or ameliorate at least one symptom of the ocular disease.

18. The composition according to claim 17, wherein the drug is at least one of beta blocker, carbonic anhydrase inhibitor, prostaglandin analogue, miotic, and sympthaomimetic and the efflux transporter inhibitor is at least one of bevacizumab, itraconazole, caroxyamidotriazole, TNP-470, CM101, IFN-α, IL-12, platelet factor-4, suramin, SU5416, thrombospondin, VEGFR antagonists, angiostatic, steroids+heparin, cartilage-derived Angiogenesis Inhibitory Factor, matrix metalloproteinase inhibitors, angiostatin, endostatin, 2-methoxyestradiol, tecogalan, tetrathiomolybdate, thalidomide, thrombospondin, prolactin, αᵥβ₃ inhibitors, linomide, tasquinimod, and cyclosporine A.
